(19) United States
(12) Patent Application Publication
(10) Pub. No.: US 2010/0166851 A1
(43) Pub. Date: Jul. 1, 2010

(54) Composition having lipolytic activity, production method thereof and use of the composition

(75) Inventor: Constantin Dallas, Beziers (FR)

Correspondence Address: THE NATH LAW GROUP, 112 South West Street, Alexandria, VA, 22314, US

(73) Assignee: NUTRACEUTIC ET BUSINESS CONSULTING, Beziers (FR)

(21) Appl. No.: 12/451,248

(22) PCT Filed: Jun. 29, 2007
(86) PCT No.: PCT/FR2007/051559

§ 371 Date: Mar. 8, 2010

(30) Foreign Application Priority Data
May 4, 2007 (FR) 0754856

(51) Int. Cl. A61K 31/522; A61K 9/48

(52) U.S. Cl. 424/451; 514/263.31

(57) ABSTRACT

The invention relates to a composition having lipolytic activity that comprises at least polyphenols, especially in the form of flavonoids, in particular flavanones and anthocyanins, and phenolic acids, and caffeine. The invention also relates to a process for obtaining such a composition and the use of the composition for its administration with a view to promoting the burning of fats or promoting the loss of body mass.

The invention relates to a composition having lipolytic activity, namely obtained from citrus fruits.

The invention also relates to a method for obtaining such a composition as well as to the use of such a composition.

The invention is related to the field of food complements and powders for their food application, i.e. namely their incorporation into food.

The lipolysis is the biological phenomenon corresponding to the breakdown of fat by the organism.

Such a breakdown can namely permit to release energy accumulated in the form of fat in the fatty tissue. At physiological level, this breakdown occurs namely at the level of the adipocytary cells, in the form of a reaction consisting in releasing fatty is acids and glycerol through hydrolysis of the triglycerides accumulated in said adipocytary cells.

Thus, enhancing the lipolysis phenomenon is an efficient solution for inducing loss of body mass and body strengthening assisted by muscle power training, since the lipolysis results into burning fat and can thus permit to increase the physical performances.

Some food complements are known for their action in this direction, but they incorporate synephrine, an alkaloid structurally similar to ephedrine. Unwanted effects at cardiovascular and cerebro-vascular levels have been reported when using compositions comprising these molecules.

Other molecules are also known for their potentially, but not exclusively, lipolytic activity. These are for example theophylline, caffeine, or also isoproterenol. Though theophylline and caffeine are widely known for being naturally present in plants such as tea or coffee, they are also excitatory substances the excess of which is not compatible with a healthy and calibrated food diet. Isoproterenol is a medicinal substance.

The present invention pretends to be capable of meeting the expectations of the consumers looking for a food complement or powder for its food application having lipolytic activity, which is of natural origin, efficient while remaining free of any unwanted side effect.

Studies carried out on extracts of fruit, namely citrus fruit, have allowed to put forward the unique lypolitic action of a composition according to the invention.

To this end, the invention relates to a composition having lipolytic activity, wherein it comprises at least polyphenols, namely in the form of flavonoids, in particular flavanones and anthocyanins, and phenolic acids, and caffeine.

The composition according to the invention may also comprise, in addition, C-vitamin.

According to another feature of the invention, the composition according to the invention comprises at least 30% polyphenols, preferably between 60 and 90%.

According to another feature of the invention, the composition according to the invention comprises 0.1 to 4% caffeine.

According to another feature of the invention, the composition according to the invention comprises 0.1 to 3% C-vitamin.

According to another feature of the invention, the composition according to the invention comprises at least 0.1 to 30% flavanones, 0.01 to 10% anthocyanins and 0.01 to 5% phenolic acids, the percentages relating to the total composition.

Advantageously, the composition according to the invention is obtained from a mixture of one or several plant species among blood orange (Citrus sinensis L. Osbeck), sweet orange (Citrus aurantium L. var. sinensis L.), bitter orange (Citrus aurantium L. var. amara), grapefruit (Citrus paradisi), guarana (Paulinia cupanna), mate (Illex Paraguariensis), cola nut (Cola Nitida).

According to another feature of the invention, the composition according to the invention is obtained from a mixture of plant species including at least 5 to 30 wt % blood oranges (Citrus sinensis L. Osbeck), 5 to 30 wt % grapefuits (Citrus paradisi), 5 to 25 wt % sweet oranges (Citrus aurantium L. var. sinensis L.), 1 to 10 wt % bitter oranges (Citrus aurantium L. var. amara), and 5 to 20 wt % guarana (Paulinia cupanna).

Advantageously, the composition according to the invention is alone or with additional additives, in the form of a water-soluble or water-insoluble powder. This powder can be integrated in various galenic forms or used alone in food products.

The invention also relates to a method for obtaining such a composition according to the invention, wherein the method includes at least:
a step of extracting polyphenols from blood orange (Citrus sinensis L. Osbeck) and sweet orange juice (Citrus aurantium L. var. sinensis L.),
a step of extracting polyphenols from by-products of blood orange (Citrus sinensis L. Osbeck), grapefruit (Citrus paradisi), sweet orange (Citrus aurantium L. var. sinensis L.) and bitter orange (Citrus aurantium L. var. amara),
a step of mixing the extracts obtained from the preceding steps of extraction,
after the step of mixing, a step of drying the mixture through atomizing on a maltodextrin carrier,
after the step of drying, a step of normalizing in caffeine, namely through addition of an extract of guarana (Paulinia cupanna), and/or mate (Illex Paraguariensis), and/or cola nut (Cola Nitida) to the product resulting from the step of drying,
then, finally, a step of sieving the resulting product, in order to obtain a powder.

According to another feature of the invention, the method is characterized in that the extraction of anthocyanins and phenolic acids from blood orange (Citrus sinensis L. Osbeck) and sweet orange juice (Citrus aurantium L. var. sinensis L.) and the extraction of flavanones from by-products of blood orange (Citrus sinensis L. Osbeck), grapefruit (Citrus paradisi), sweet orange (Citrus aurantium L. var. sinensis L.) and bitter orange (Citrus aurantium L. var. amara) are proceeded to.

The invention also relates to the use of the composition according to the invention as a food complement or for enriching food, namely beverages such as fruit and/or vegetable juices as well as the milk products.

Advantageously, the composition according to the invention is used for its administration with a view to enhance the breakdown of fat or enhance the loss of body mass.

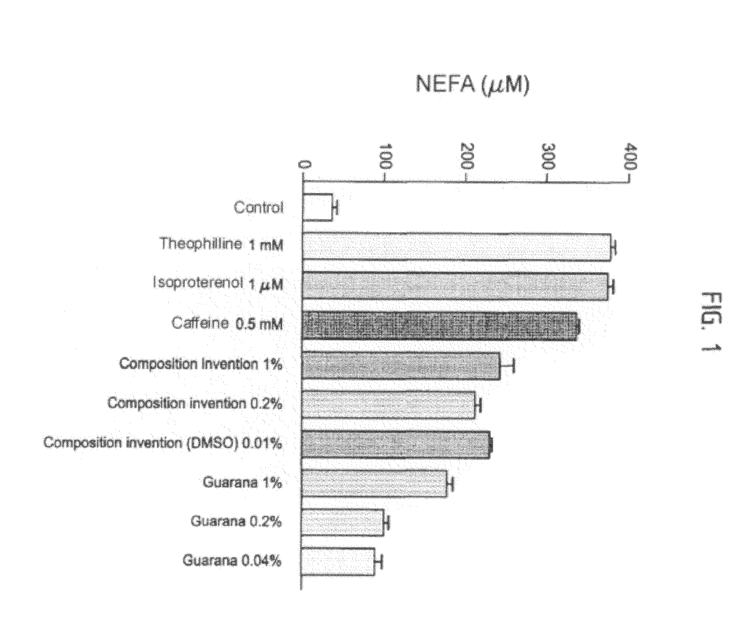

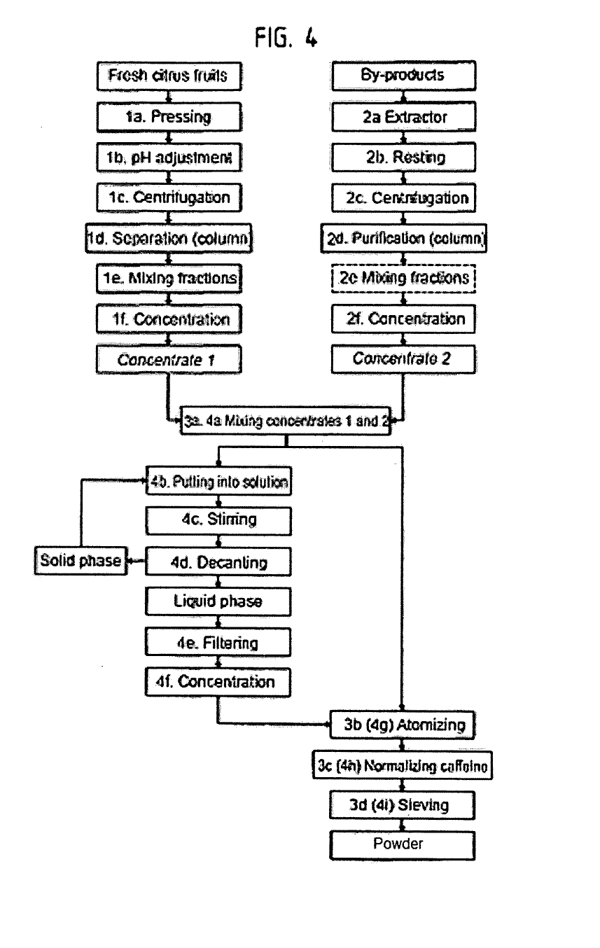

The aims and advantages of the present invention will clearly appear from the following detailed description relating to several exemplary embodiments. This description will be better understood with reference to the attached drawings, in which FIGS. 1 to 3 show experimental results proving the efficiency of the composition according to the invention, and FIG. 4 is a diagram showing an embodiment of the method for obtaining the composition according to the invention.

The present invention is related to the field of the food complements and powders for their food application, i.e. namely their incorporation into food, and relates in particular to a composition having lipolytic activity, namely obtained from citrus fruits.

The plant species for obtaining same have been chosen so as to obtain a composition providing an adequate dose of active substances acting in synergy.

In particular, the composition according to the invention makes use of a synergy between the effects of the polyphenols, namely in the form of flavonoids and phenolic acids, and caffeine it contains.

Advantageously, the synergy is increased by the presence in addition of C-vitamin.

The polyphenols are more exactly to be found in the form of flavonoids and phenolic acids. In particular, the composition according to the invention has particular flavanone and anthocyanin contents, which are both subgroups of flavonoids, as well as particular phenolic acid contents.

Table 1 shows the dose of these various essential active substances in a preferred embodiment of the composition according to the invention.

In particular, the composition having lipolytic activity according to the invention comprises at least 30% polyphenols, preferably between 60 and 90%, and 0.1 to 4% caffeine.

The polyphenols are in particular represented by flavanones, anthocyanins and phenolic acids. Advantageously, the composition comprises in particular 0.1 to 30% flavanones, 0.01 to 10% anthocyanins and 0.01 to 5% phenolic acids, the percentages relating to the total composition and the total polyphenols present reaching at least 30% of the total composition.

The flavanones can be neither exclusively nor exhaustively, but dominantly, for example in the form of hesperidin, naringin, isonaringin, narirutine, neohesperidin, naringenin.

The anthocyanins can be neither exclusively nor exhaustively, but dominantly, for example in the form of cyanidin-3-glucoside, malvidin-3-glucoside, peonidin-3-glucoside, petunidin-3-glucoside.

The phenolic acids can be neither exclusively nor exhaustively, but dominantly, for example in the form of caffeic acid, coumaric acid, ferulic acid, cinammic acid, sinapic acid.

The C-vitamin is advantageously present at a level of 0.1 to 3% of the total composition.

The percentages are calculated from the analysis of a sample of the composition according to the invention by various methods. The total polyphenols are measured by ultraviolet spectrophotometry at 280 nm, and the anthocyanins by the same technique at 550 nm. The flavanones, phenolic acids, also the anthocyanins, caffeine and C-vitamin are, in turn, measured by High Performance Liquid Chromatography (HPLC).

An optimized method, which we will describe hereafter, permits to obtain the composition having lipolytic activity according to the invention, which is finally in the form of a water-soluble or water-insoluble powder.

Surprisingly, experiments have recognized the dose of active substances presented above as particularly efficient to meet the objectives sought, i.e. an effect on the breakdown of fat and, thus, in the increase of the physical performances.

In particular, the composition according to the invention acts on the lipolysis.

For a better understanding of these experiments, it is necessary to previously set forth the biologic factors involved in the lipolysis.

The lipolysis is a reaction consisting in releasing fatty acids and glycerol through lipid hydrolysis. This can occur at the level of the digestion of the food lipids, but also at the level of the fatty tissue, or the peripheral lipolysis reaction consists in releasing fatty acids and glycerol through hydrolysis of the triglycerides accumulated in the adipocytary cells of the organism. This is the reaction we are particularly interested in, corresponding to the expected effect of breakdown of the accumulated fat.

The lipolysis is a hormonosensitive reaction, namely sensitive to the hormones <<catecholamines>> such as adrenaline or noradrenaline. It is triggered by the link of these hormones to the β-3 receptors of the adipocytary cells, which results into an activation of the adenyl-cyclase, enzyme of the cyclic AMP synthesis (cAMP), which thus causes an intracellular increase in cyclic AMP (cAMP). The cyclic AMP (cAMP) is a molecule activating the triglyceride lipase, which is in turn responsible for the release of reserve fat accumulated in the form of triglycerides in the adipocytary cells. The release of the reserve fat occurs through hydrolysis of the triglycerides, according to the reaction:

1 triglyceride→3 Free Fatty Acids (FFA)+1 Glycerol

The FFA released then act as an energetic substrate for the cells of the organism.

The lipolysis inhibition, which induces the effect inverse to the expected one, can namely be brought about by the action of two enzymes: cathechol-o-methyl transferase and phosphodiesterase. The cathechol-o-methyl transferase intervenes in the catabolism of the hormones that link to the β-3 receptors, and results into limiting the stimulation of the cycle resulting into the release of FFA. The phosphodiesterase intervenes in the breakdown of the cyclic AMP (cAMP) into an inactive non-cyclic form, which also results into limiting the stimulation of the cycle resulting into the release of FFA.

The experiments that have been conducted in order to test the efficiency of the composition according to the invention consist, on the one hand, of an ex-vivo experiment conducted on human adipocytes, in order to assess the effects of the composition according to the invention on the release of FFA, also referred to as Not Esterified Fatty Acids (NEFA), this compared to other substances, and, on the other hand, of an in-vivo experiment, i.e. a clinical study carried out on overweight individuals and to whom the composition according to the invention has been administered, compared to the administration of a placebo.

These two experiments are particular revealing as to the physiological effects of an administration of the composition according to the invention.

Experiment 1

A first experiment, the results of which are shown in table 2 and in FIG. 1, was aimed at detecting the effects of an administration of the composition according to the invention on the release of Not Esterified Fatty Acids (NEFA) by human adipocytes maintained in suspension. This measurement is indeed revealing as to the effect of the composition according to the invention on the hydrolysis of the adipocytary triglycerides.

This experiment has been conducted on human adipocytes isolated from an abdominal plasty of a female donor, aged 35. Immediately upon receipt, the fatty tissue fragments have been incubated for 30 minutes at 37° C. in the presence of collagenase. The isolated and washed adipocytes have been mixed with 4 volumes of test medium (bicarbonate 1.87 mg/mL, penicillin/streptomycin 25 UI/mL/25 μg/mL, glutamine 2 mM, MEM without phenol red qsq 100% (v/v), to which was added delipidated bovine seric albumin at 0.5% (w/v)).

In order to obtain comparative results, various compounds have been tested in parallel on the adipocytes in suspension before measuring the NEFA, i.e. solutions of theophylline, isoproterenol, caffeine, guarana titrated 12% with caffeine and finally a solution of the composition according to the invention.

To 540 uL of a suspension of adipocytes in a test medium milieu have been added 60 μL of compound to be tested, and the mixtures have been incubated at 37° C., under stirring, for 2 hours.

A final concentration of theophylline in the tested mixture of 1 mM was maintained, starting from a stock solution of 10⁻² M in test medium.

Likewise, a final concentration of isoproterenol in the tested mixture of 1 μM was maintained, starting from a stock solution of 10⁻² M in test medium.

A final concentration of caffeine in the tested mixture of 0.5 mM was also maintained, starting from a stock solution of 5 mM in test medium.

Since the guarana titrated 12% in caffeine is supplied in the form of a powder, a stock solution was prepared at 20 mg/mL, i.e. 2% (w/v), in test medium. Starting from this stock solution, the tests were carried out on three final dilutions of the stock solution in the tested mixture, 1%, 0.2% and 0.04%.

Since the composition according to the invention is a powder, it was diluted in DMSO, on the one hand, and in test medium, on the other hand. The stock solution prepared with DMSO was prepared at 10 mg/mL, i.e. 1% (w/v), for a final concentration of the composition according to the invention in solution in DMSO in the tested mixture of 0.01%.

The stock solution prepared in test medium was prepared at 20 mg/mL, i.e. 2% (w/v), for a final concentration of the composition according to the invention in solution in test medium in the tested mixture of 1% and 0.2%.

A control comprising only the adipocytes in test medium was also prepared.

A total of 10 doses were prepared from 10 different fractions with the compound added or not.

At the end of the incubation time, the NEFA were measured into fractions of 30 μL of medium, after decanting for 5 minutes. The dose was prepared conventionally by means of a NEFA-C measuring kit.

The measured data were processed statistically. Intergroup comparisons were carried out through variance analysis (ANOVA) by means of the Dunnet multiple comparison test.

The results of this study are given in table 2 and FIG. 1, the latter consisting of is a graphic representation of the results present in table 2.

The basal lipolysis, as shown by the control, was of 36 μM NEFA released in 2 hours.

The theophylline at 1 mM and the isoproterenol at 1 μM stimulated the lipolysis considerably (respectively a factor 10 with respect to the control, p<0.01). The caffeine tested at 0.5 mM clearly stimulated the release of NEFA (factor about 9.5 with respect to the control, p<0.01). Thus, the lipolysis stimulations obtained with these compounds, which are reference compounds having known effects, validate the experiment of release of Not Esterified Fatty Acids (NEFA).

The addition of composition according to the invention solubilized in aqueous test medium, tested at 1% and 0.2%, results into a clear stimulation of the release of NEFA (respectively by a factor of about 7 and 6 with respect to the control). These stimulations prove to be dependent on the dose. The composition according to the invention solubilized in DMSO and tested at 0.01% stimulated the lipolysis with an activity similar to that obtained at 1% in test medium.

Finally, the dilution of guarana titrated 12% in caffeine, tested at 1%, 0.2% and 0.04% stimulated considerably and depending on the dose the lipolytic activity (respectively by a factor 5, 3 and 2.5 with respect to the control).

Furthermore, controls of absence of interference of the product with the dose of NEFA were performed systematically. A negative control was performed by measuring the Optical Density (OD) of the fractions of compounds at the final concentrations used (same medium, but without adipocytes). The presence of compound should not considerably increase the signal, i.e. the presence of compound should not considerably change the OD of the sample, which can be due to a high OD of the compound at the final dilutions used or the presence of a non-negligible quantity of Fatty Acids in the compound under test. A positive control was performed by measuring a known quantity of NEFA (0.5 mM) in the presence or the absence of the compound under test. The presence of the product should not notably decrease the signal.

In the conditions of experiment set forth, the compounds did not exhibit any interferences with the dose of NEFA for any of the controls. Only a slight interaction was observed with the composition according to the invention diluted in DMSO, where the positive control showed a slight interaction (underestimated dose of NEFA), but this interference does not disturb the interpreting of the results.

As a conclusion of this experiment, it seems that at an equivalent dose, in the conditions of the experiment, the composition according to the invention has a lipolytic activity higher than that of guarana, showing without any ambiguity the synergy of the components it is comprised of for stimulating the ex-vivo lipolysis in human adipocytes.

Experiment 2

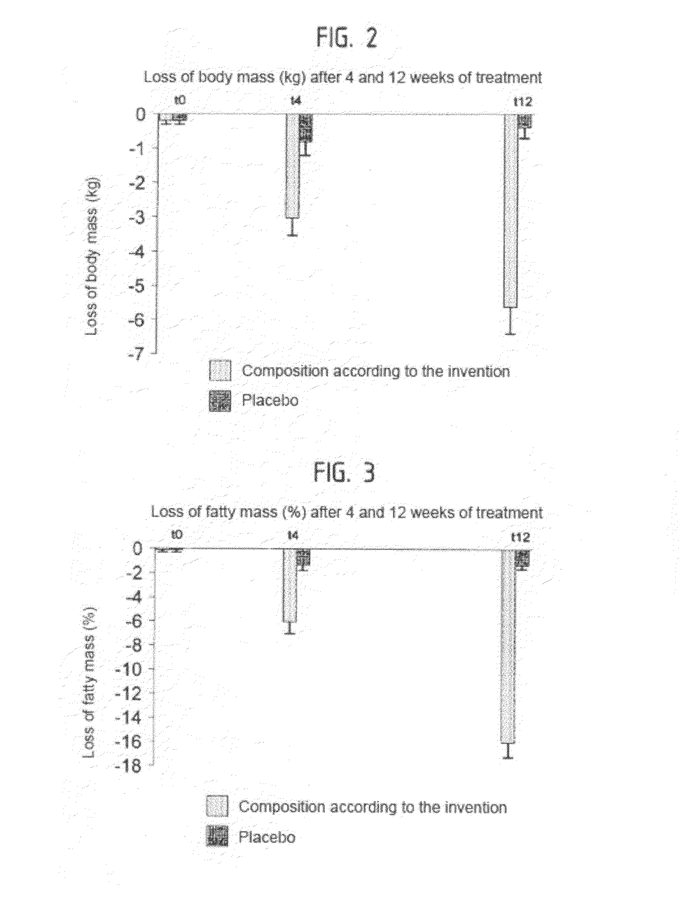

The second experiment consists of a clinical study the results of which are given in tables 3 to 6 and FIGS. 2 and 3. This study was aimed at detecting the effects of an administration of the composition according to the invention on overweight individuals, compared to the administration of a placebo. Two variables have been checked, namely the body mass and the percentage of the fatty mass.

These variables are indeed macroscopic indicators of the action of the composition according to the invention on the activity of breakdown of fat following the lipolysis and the resulting loss of body mass.

The clinical study was performed on 20 individuals distributed into two groups:
A group referred to as ‘placebo’ of 10 persons (9 women and 1 man), having a Body Mass Index (BMI) between 27 and 30 for an average BMI of 28.5. The age of the individuals in this group is between 22 and 55 years and the average body mass observed at the beginning of the study was 73 Kg.
A group referred to as ‘composition according to the invention’ of 10 persons (7 women and 3 men), having an average BMI of 28.1. In particular, two subgroups were distinguished, the first subgroup A (4 women) being formed of individuals considered obese, with a BMI between 29 and 33, the second subgroup B (3 women, 3 men) being formed of individuals considered overweight, with a BMI between 27 and 30. The age of the individuals is between 25 and 55 years and the average body mass observed at the beginning of the study was 70.5 Kg.

The BMI is an index that is very often used in the studies on obesity, corresponding to the body mass of a person in Kg, divided by its size in square meters (BMI=Kg/m²).

Despite their overweight, the individuals of the study were chosen without any health problems (total exclusion on hepatic, cardiovascular or kidney dysfunctions), without pregnancy, non-smokers, and non-consumers of medicines or any other food supplements.

The individuals have been followed up for 12 weeks, during which the composition according to the invention was administered to the group ‘composition according to the invention’ in the form of 4 capsules each containing 350 mg of power of the composition according to the invention, 2 capsules in the morning and 2 capsules during the main meal. The group ‘placebo’ received in the same forms a placebo.

The individuals maintained their quantity of usual physical exercise previous to the study as well as their food habits (between 1500 and 2000 calories a day).

Periodic measurements of the percentage of fatty mass of the individuals through bioelectric impedance analysis and of their body mass through weighing were performed during these 12 weeks.

Tables 3 and 4 show the data corresponding to the measurements of body mass, respectively for the individuals of the group ‘placebo’ and ‘composition according to the invention’.

It clearly appears that, though the loss of body mass is insignificant, individually or as an average, for the individuals of the group ‘placebo’, the individuals of the group ‘composition according to the invention’ saw their body mass decrease as an average by 3 Kg at week 4 and 5.6 Kg at week 12.

FIG. 2 shows these results by showing in the form of a graph the evolution of the average body mass in the two groups, namely at weeks 4 and 12.

In particular, the individuals of the subgroup A, who had in the beginning an important BMI between 29 and 33, showed the largest loss of body mass at the end of the 12 weeks of the study.

Tables 5 and 6 show the data corresponding to the measurements of fatty mass, respectively for the individuals of the group “placebo’ and ‘composition according to the invention’.

It clearly appears that, though the loss of fatty mass is insignificant, individually or as an average, for the individuals of the group ‘placebo’, the individuals of the group ‘composition according to the invention’ saw their fatty mass decrease as an average by 5.9% at week 4 and 15.6% at week 12.

FIG. 3 shows these results by showing in the form of a graph the evolution of the average fatty mass in the two groups, namely at weeks 4 and 12.

In particular, the individuals of the subgroup A, who had in the beginning an important BMI between 29 and 33, showed the largest loss of fatty mass at the end of the 12 weeks of the study.

No major side effect, such as tachycardia or blood-pressure drop, was observed in any of the individuals during the 12 weeks of treatment with 1.4 grams a day of the composition according to the invention.

It appears from this clinical study that the composition according to the invention proves both sure and efficient as regards its effects related to the breakdown of fat and to loss of body mass.

In order to obtain the composition according to the invention, there was proceeded to a selection of plants, namely citrus fruits, the combination of which is particularly suited for obtaining said composition. Table 7 shows the necessary essential plant species, according to a preferred embodiment, where the composition is obtained from a mixture of blood oranges, sweet oranges, bitter oranges, grapefruits and, preferably guarana. A weight percentage fixing the representation of each plant species is advantageously stuck to.

The normalizing in caffeine can, according to further embodiments, however be obtained from plant species such as mate (Illex Paraguariensis) or cola nut (Cola Nitida), according to any combinations between these species and with the guarana.

The chosen blood orange varieties are advantageously from the following list: Citrus sinensis L. Osbeck CV ‘Sanguinelli’, Citrus sinensis L. Osbeck CV ‘Tarocco^{r}, Citrus sinensis L. Osbeck CV ‘Melitense’, Citrus sinensis L. Osbeck CV ‘St Michael’.

The natural origin of the composition according to the invention and its components explain the double action mechanism that permits it to potentiate the action of the lipolysis in adipocytes. It should be reminded that the composition according to the invention is completely synephrine-free.

The polyphenolic structure of most of its components permits to inhibit the action of cathechol-o-methyl transferase, while its unique association of polyphenols and xanthic bases permits to inhibit the action of phosphodiesterase.

By way of comparison, the action of theophylline is known to be exerted exclusively on cathechol-o-methyl transferase and the action of caffeine on phosphodiesterase.

Finally, the composition according to the invention is obtained thanks to a preferred method, the steps of which are transcribed below. FIG. 4 shows furthermore, by means of a diagram, the detailed sub-steps of an embodiment of said method.

This method is fully suited for obtaining a composition according to the invention. It is namely based on two fundamental points: on the one hand, it permits to selectively extract a set of polyphenols (belonging to several different subgroups as set forth in detail above), having synergically a lipolytic activity, on the other hand, it ensures the absence of synephrine in the final composition, thanks to a step of selective purification on column.

In particular, as regards the selective extraction of the polyphenols entering into the composition according to the invention, it was necessary to carry out a mixture of plant species, namely citrus fruits, to obtain the composition according to the invention, since not all the polyphenols sought are present in one single plant species, but in several of them. In addition, the various polyphenols are dominantly present either in the juice or in the peels and/or seeds of the plant species previously selected according to the preceding rule, whence the necessity of carrying out a double extraction namely from the juice and from the peels.

Advantageously, the composition according to the invention can also be obtained in the form of a water-soluble or water-insoluble powder.

For a better disclosure of the method for obtaining the composition according to the invention, four different steps will be explained:
a first step, called step 1, consists in extracting polyphenols, namely anthocyanins and phenolic acids, from the juices of the selected citrus fruits;
a second step, called step 2, consists in extracting polyphenols, namely flavanones, from the peels and/or seeds of the selected citrus fruits;
a third step, called step 3, permits, after the mixing of the products obtained at the end of the steps 1 and 2, respectively, to obtain a powder of the composition according to the invention, said powder being water-insoluble;
a fourth step, called step 4, permits, after the mixing of the products obtained at the end of the steps 1 and 2, respectively, to obtain a powder of the composition according to the invention, said powder being water-soluble;

It thus appears that the steps 1 and 2 are prior to the steps 3 and 4, whereby the steps 1 and 2 can however be carried out in parallel. The steps 3 and 4 are two alternative embodiments for obtaining a powder of the composition according to the invention, the step 4 differing from step 3 by the addition of additional steps to the method for obtaining a powder according to step 3.

Hereafter is described an embodiment of said method according to the invention, in which the sub-steps constituting said method are more particularly detailed. FIG. 4 shows by means of a diagram the chronology of these sub-steps while indicating their reference numerals (the reference numerals are indicated in brackets in the present description).

Step 1

The first step consists in extracting polyphenols from the juices of the selected citrus fruits. In particular, the citrus fruits used are blood oranges (Citrus sinensis L. Osbeck) and sweet oranges (Citrus aurantium L. var. sinensis L.). Fresh citrus fruits will preferably be used. It is also possible to use dried or deep-frozen citrus fruits.

The anthocyanins belong to the family of the polyphenols and are characterized by the presence of two benzenic cycles that surround a pyrylium cycle with a catenation of carbons C3-C6-C3. In the blood orange (Citrus sinensis L. Osbeck) the anthocyanins are in the form of sugars (monosaccharides such as glucose). The glycosilation occurs in position 3 of the anthocyanidines (aglycone form). The dominant anthocyanin in the blood orange (Citrus sinensis L. Osbeck) is the Cyanidin-3-glucoside. The phenolic acids are also present in the juices of the citrus fruits.

Thus, the step 1 permits in particular the extraction of the anthocyanins and phenolic acids from these citrus fruits.

C-vitamin C is also present in the juices of the citrus fruits, and extracted at the same time.

The selected citrus fruits are pressed in cold (1a), individually or combined into different groups.

The pH of the juices obtained by pressing is immediately brought (1b) to a value between 1.5 and 2 through addition of hydrochloric acid (HCl) 0.1% 12 M or formic acid 10%. A low pH value is necessary to maintain the anthocyanins essentially in the form of flavylium cation (most stable form). A previous study has shown that 97% of the anthocyanins are in the form of flavylium cation at a pH between 1.5 and 2, and only 55% when the pH value is higher than 2.5.

Then, the juices are centrifuged (1e) for 30 minutes to 3 hours at 4000 rev/min, and the liquid phase collected with a view to purification by separation (1d) on column.

A double elution with different solvents is carried out conventionally, in order to namely eliminate the synephrine (since the synephrine is completely eliminated within the framework of the composition according to the invention). The anthocyanins and phenolic acids are capable of being reversibly fixed on a large number of absorbent columns, thus permitting to namely eliminate the synephrine. The column used can advantageously be comprised of a mixture of polyvinylpyrrolidone (pvp) and gel (Sephadex G25) in a respective ratio from 70:30 to 95:5. Other types of columns can obviously be chosen, even though this carrier mixture proved the most efficient one for a good separation, thanks to its good retaining of anthocyanins and phenolic acids when the juice passes through the column.

A fractioning into two parts is obtained depending on the elution solvents used. A first fraction is obtained after elution of the column with water, the latter carrying along the sugars, the acids and the soluble polysaccharides in the first fraction. A second fraction is obtained after elution with a mixture of solvents Ethanol/Water/Formic acid (in proportions 30:68:2 to 83:15:2, respectively), permitting to obtain mainly the anthocyanins-3-monoglycosides and phenolic acids in the second fraction.

The control of these two fractions by HPLC analysis showed the absence of interfering substances such as synephrine or octopamine.

The, the resulting fractions are mixed (1e) and the mixture is concentrated (1f) under vacuum, which permits to eliminate all the solvents used and a large portion of the water, to obtain a concentrate that we will call concentrate 1.

Though 50% of the mass of the citrus fruits is formed by their juice, from this only exploitation results an important quantity of by-products (mainly from the peels and seeds). These by-products will thus be used in step 2.

Step 2

The second step consists in extracting polyphenols from the by-products of the selected citrus fruits. In particular, the citrus fruits used are blood oranges (Citrus sinensis L. Osbeck), grapefruits (Citrus paradisi), sweet oranges (Citrus aurantium L. var. sinensis L.) and bitter oranges (Citrus aurantium L. var. amara).

By-products of fresh citrus fruits will preferably be chosen, but these may also be dried or deep-frozen.

Thus, step 2 permits in particular the extraction of the flavanones from these citrus fruits.

A mixture of by-products of blood orange (Citrus sinensis L. Osbeck), grapefruit (Citrus paradisi), sweet orange (Citrus aurantium L. var. sinensis L.) and bitter orange (Citrus aurantium L. var. amara) is placed in an extractor (2a) for 1 to 12 hours at 40-70° Centigrade in the presence of solvents, for example a mixture of Ethanol/Methanol/Water in the respective proportions 40:10:50.

The mixture may then rest (2b) in the extractor for 4 to 6 hours.

The, the content of the extractor is centrifuged (2c) for 30 minutes to 3 hours at 4000 rev/min. The solid phase is separated and the liquid phase is introduced into a purification column (2d). The latter is advantageously, but not exclusively a resin column. The flavanones are obtained after elution of the column with a mixture of solvents Ethanol/Methanol/Water in respective proportions of 65:5:30.

The resulting final extract, if necessary after mixing (2nd step optional) of various extracts proceeding from the preceding steps, is then concentrated (2f) under vacuum, to obtain a concentrated that we will call concentrate 2.

Step 3

The third step consists in mixing (3a) the two concentrates 1 and 2 with a view to obtain a powder. Thus, the mixed extracts 1 and 2 are dried through atomizing (3b) on a maltodextrine carrier. The product obtained is normalized in caffeine (3c), by mixing with a guarana extract (Paulinia cupanna) titrated 12% in natural caffeine, in the ratios Composition according to the invention/guarana extract of 80:20 to 95:5. The final gross caffeine content then varies between 0.1 and 4% of the composition according to the invention. Other conventional sources of caffeine may be used to achieve this objective, as for example the use of mate or cola nut. The end product is sieved (3d), in order to obtain a powder of the composition according to the invention that is water-insoluble, with a grain size between 40 and 100 mesh.

Step 4

The fourth step consists in mixing (4a) the two concentrates 1 and 2 with a view to obtain a water-soluble powder. Thus, the mixed extracts 1 and 2 are put into solution with water (4b), in a ratio of one unit of mixture of the two concentrates for 10 units of water, and brought to 20° Centigrade for 1 to 5 hours under stirring (4c) and in an inert atmosphere.

Then the separation of the non-soluble particles by decanting (4d) for 1 to 3 hours is proceeded to. The surface fraction is then taken. The non-soluble deposit is treated again with water in a new sepaLtion identical to that described before (4b to 4d).

The recovered liquid fractions are then filtered (4e) on filtering paper. The final ratio of the two Insoluble/Soluble fractions is between 15 and 30% of the insoluble fraction for 85 to 70% of the soluble fraction, respectively.

Then the water-soluble fractions are submitted to the same treatment as described in step 3, i.e. concentrated (4f) under vacuum and dried through atomizing (4g) on maltodextrin.

The product obtained is normalized in caffeine (4h), by mixing with a guarana extract (Paulinia cupanna) titrated 12% in natural caffeine, in the ratios Composition according to the invention/guarana of 80:20 to 95:5. The final gross caffeine content then varies between 0.1 and 4% in the composition according to the invention. Other conventional sources of caffeine may be used to reach this objective, as for example the use of mate or cola nut. The end product is sieved (4i), in order to obtain a water-soluble powder of the composition according to the invention, with a grain size between 40 and 100 mesh.

Through the method according to the invention it is thus possible to obtain a composition that advantageously solves the problem set forth, i.e. a composition having a lypolitic activity permitting to encourage the loss of body mass and the body strengthening assisted by muscle power training, since the lipolysis results into the breakdown of fat and can permit to increase the physical performances. The composition obtained is in addition fully synephrine-free.

The powder obtained at the end of the method according to the invention can be water-soluble.

The invention also relates to the use of the composition obtained as food complement or to enrich food.

Its use as food complement will be facilitated in its form of water-insoluble powder. In this context and non-exhaustively, the water-insoluble powder obtained can be used, alone or with additional additives, in galenic form, namely of capsulated powder, for example in capsule, of powder compressed into tablets or granulates of any shape.

Its use to enrich food will be facilitated in its form of water-soluble powder. The water-soluble powder can indeed advantageously be used, alone or with additional additives, for example being dissolved in a food liquid, and thus incorporated to beverages, or in milk products such as yoghurts. It can also be incorporated, non-exhaustively, into biscuits or confectionery, or presented in the form of water-soluble bags.

The beverages may, non-exhaustively, be for example fruit and/or vegetable juices.

The invention also relates to the use of the composition obtained for its dietetic administration.

Finally, it should be reminded that the administration of the composition according to the invention advantageously permits to enhance the breakdown of fat or the loss of body mass. It is particularly efficient in individuals having a BMI of the obese type, i.e. higher than 30.

The posology studied corresponds to the preferred posology, which is of about 1.4 grams per day of composition. This posology can of course be adapted according to the physiological response to the treatment, and vary between 1.0 and 2.0 grams per day and per individual.

TABLE 1
Essential Active Substances

| | | % of the total |
|---|---|---|
| Active substances | | composition |
| 1) Total polyphenols | | 30-100% |
| Flavonoids | Flavanones | 0.1-30% |
| | i.e Hesperidin, Naringin, | |
| | Isonaringin, Narirutine, | |
| | Neohesperidin, Naringenin . . . | |
| | Total Anthocyanins | 0.01-10% |
| | i.e Cyanidin-3-glucoside, Malvidin- | |
| | 3-glucoside, Peonidin-3-glucoside, | |
| | Petunidin-3-glucoside . . . | |
| | Phenolic Acids | 0.01-5% |
| | i.e Caffeic acid, Coumaric acid, | |
| | Ferulic acid, Cinnamic acid, Sinapic | |
| | acid . . . | |

| | |
|---|---|
| 2) Caffeine | 0.1-4% |
| 3) C-Vitamin | 0.1-3% |

TABLE 2
EXPERIMENT 1 - Dose of released NEFA

| | | NEFA | | | % of the | |
|---|---|---|---|---|---|---|
| Treatment | Concentration | (μM) | sd | n | control | P |
| Untreated control | — | 36 | 10 | 3 | 100 | — |
| Theophylline | 1.0 mM | 381 | 9 | 3 | 1057 | p < 0.01 |
| Isoproterenol | 1.0 μM | 377 | 11 | 3 | 1048 | p < 0.01 |
| Caffeine | 0.5 mM | 339 | 4 | 3 | 941 | p < 0.01 |
| Composition according to | 1.0% | 244 | 17 | 3 | 677 | p < 0.01 |
| the invention in test medium | 0.2% | 213 | 13 | 3 | 592 | p < 0.01 |
| Composition according to | 0.01% | 231 | 6 | 3 | 643 | p < 0.01 |
| the invention in DMSO | | | | | | |
| Guarana 12% | 1.0% | 178 | 15 | 3 | 494 | p < 0.01 |
| | 0.2% | 101 | 11 | 3 | 281 | p < 0.01 |
| | 0.4% | 90 | 17 | 3 | 249 | p < 0.01 |

sd: standard difference
n: number of observations
p: result of the variance analysis

TABLE 3
EXPERIMENT 2 - Evolution of the body mass after
administration of a placebo

| | | | | Body | Loss of | Loss of |
|---|---|---|---|---|---|---|
| | | | | mass | body mass | body mass |
| | Age | Sex | BMI | t0 (Kg) | t4 (Kg) | t12 (Kg) |
| Individual 1 | 22 | F | 28.8 | 71.9 | −1.4 | −1.4 |
| Individual 2 | 33 | F | 28.5 | 70.2 | −0.8 | −1.3 |
| Individual 3 | 29 | F | 28.2 | 71.4 | −0.5 | −0.4 |
| Individual 4 | 25 | F | 29.3 | 67.7 | −0.9 | +2.9 |
| Individual 5 | 55 | F | 28.2 | 74.1 | −0.2 | −0.2 |
| Individual 6 | 45 | F | 26.9 | 68.8 | −0.7 | −0.8 |
| Individual 7 | 28 | F | 29.1 | 78.4 | −1.2 | −1.0 |
| Individual 8 | 47 | M | 28.2 | 84.6 | −0.9 | −0.7 |
| Individual 9 | 25 | F | 28.4 | 72.8 | −0.8 | −0.8 |
| Individual 10 | 29 | F | 29.4 | 70.7 | −0.5 | −0.7 |
| Average | 33.1 | | 28.5 | 73 | −0.8 | −0.4 |

TABLE 4
EXPERIMENT 2 - Evolution of the body mass after administration of
composition according to the invention

| | | | | Body | Loss of | Loss of |
|---|---|---|---|---|---|---|
| | | | | mass | body mass | body mass |
| | Age | Sex | BMI | t0 (Kg) | t4 (Kg) | t12 (Kg) |
| Individual 1 | 27 | F | 26.6 | 63.8 | −2.4 | −3.6 |
| Individual 2 | 33 | M | 27.0 | 69.1 | −1.4 | −3.0 |
| Individual 3 | 35 | F | 25.1 | 60.2 | −1.7 | −3.2 |
| Individual 4* | 25 | F | 28.9 | 73.9 | −3.5 | −7.5 |
| Individual 5 | 55 | M | 26.8 | 73.0 | −3.0 | −4.1 |
| Individual 6* | 47 | F | 31.8 | 80.5 | −5.3 | −8.9 |
| Individual 7* | 22 | F | 32.7 | 78.6 | −3.2 | −8.8 |
| Individual 8 | 39 | M | 25.3 | 72.2 | −4.1 | −5.1 |
| Individual 9* | 41 | F | 29.3 | 68.7 | −3.7 | −8.5 |
| Individual 10 | 37 | F | 27.5 | 65.3 | −2.0 | −3.4 |
| Average | 36.1 | | 28.1 | 70.5 | −3.0 | −5.6 |

*Individuals belonging to the group with MBI between 29 and 33

TABLE 5
EXPERIMENT 2 - Evolution of the fatty mass
after administration of a placebo

| | | | | | | Evolution |
|---|---|---|---|---|---|---|
| | | | | Fatty | Evolution of | of the |
| | | | | mass | the fatty mass | fatty mass |
| | Age | Sex | BMI | t0 (%) | t4 (%) | t12 (%) |
| Individual 1 | 22 | F | 28.8 | 31.8 | −0.3 | −0.3 |
| Individual 2 | 33 | F | 28.5 | 33.9 | −0.4 | −0.5 |
| Individual 3 | 29 | F | 28.2 | 32.5 | −0.4 | −0.5 |
| Individual 4 | 25 | F | 29.3 | 31.8 | −0.9 | +0.1 |
| Individual 5 | 55 | F | 28.2 | 32.5 | −0.4 | −0.1 |
| Individual 6 | 45 | F | 26.9 | 29.8 | −0.5 | −0.3 |
| Individual 7 | 28 | F | 29.1 | 31.5 | −0.6 | −0.3 |
| Individual 8 | 47 | M | 28.2 | 32.7 | −0.2 | −0.5 |
| Individual 9 | 25 | F | 28.4 | 31.7 | −0.2 | −1.8 |
| Individual 10 | 29 | F | 29.4 | 32.7 | −0.5 | +0 |
| Average | 33.1 | | 28.5 | 32.1 | −0.4 | −0.4 |

| | | |
|---|---|---|
| Percentage of reduction of the fatty mass | 1.25% | 1.25% |

TABLE 6
EXPERIMENT 2 - Evolution of the fatty mass after administration of
composition according to the invention

| | | | | | | Evolution |
|---|---|---|---|---|---|---|
| | | | | Fatty | Evolution of | of the |
| | | | | mass | the fatty mass | fatty mass |
| | Age | Sex | BMI | t0 (%) | t4 (%) | t12 (%) |
| Individual 1 | 27 | F | 26.6 | 28.7 | −0.8 | −2.0 |
| Individual 2 | 33 | M | 27.0 | 29.2 | −0.6 | −2.4 |
| Individual 3 | 35 | F | 25.1 | 30.1 | −0.6 | −2.0 |
| Individual 4* | 25 | F | 28.9 | 33.1 | −3.4 | −10.2 |
| Individual 5 | 55 | M | 26.8 | 28.7 | −1.2 | −2.2 |
| Individual 6* | 47 | F | 31.8 | 32.9 | −3.1 | −8.7 |
| Individual 7* | 22 | F | 32.7 | 32.2 | −2.9 | −6.8 |
| Individual 8 | 39 | M | 25.3 | 29.2 | −0.6 | −2.4 |
| Individual 9* | 41 | F | 29.3 | 33.7 | −3.5 | −9.8 |
| Individual 10 | 37 | F | 27.5 | 29.5 | −0.8 | −1.8 |
| Average | 36.1 | | 28.1 | 30.7 | −1.8 | −4.8 |

| | | |
|---|---|---|
| Percentage of reduction of the fatty mass | 5.9% | 15.6% |

*Individuals belonging to the group with MBI between 29 and 33

TABLE 7
Essential plant species

| Plant species of the | | % of the |
|---|---|---|
| composition | Botanical name | composition |
| Citrus fruits | | |
| Blood orange | Citrus sinensis L. Osbeck | 5-30% |
| Grapefruit | Citrus paradisi | 5-30% |
| Sweet orange | Citrus aurantium L. var. sinensis L. | 5-25% |
| Bitter orange | Citrus aurantium L. var. amara | 1-10% |
| Other plants | Paulinia cupana | 5-20% |
| Guarana | | |

1-22. (canceled)

23. Composition having lipolytic activity, wherein the composition comprises at least polyphenols from citrus fruits, namely in the form of flavonoids, in particular flavanones, anthocyanins, phenolic acids, and caffeine, wherein the polyphenols are represented by at least 0.1 to 30% flavanones, 0.01 to 10% anthocyanins and 0.01 to 5% phenolic acids, the percentages relating to the total composition and the total of the polyphenols present reaching at least 40% of the total composition.

24. Composition having lipolytic activity according to claim 23, wherein the composition comprises in addition C-vitamin.

25. Composition having lipolytic activity according to claim 23, wherein the composition comprises between 60 and 90%.

26. Composition having lipolytic activity according to claim 23, wherein the composition comprises 0.1 to 4% caffeine.

27. Composition having lipolytic activity according to claim 24, wherein the composition comprises 0.1 to 3% C-vitamin.

28. Composition having lipolytic activity according to claim 23, wherein the composition is, alone or with additional additives, in the form of a powder or a water-soluble powder.

29. Composition having lipolytic activity according to claim 23, wherein the composition is, alone or with additional additives, in the form of a compressed or encapsuled powder, namely in the form of capsules or tablets or granulates of any shapes or a powder bag.

30. Method for obtaining a composition having lipolytic activity according to claim 23, wherein, from a mixture of one or several plant species selected from the group consisting of blood orange (Citrus sinensis L. Osbeck), sweet orange (Citrus aurantium L. var. sinensis L.), bitter orange (Citrus aurantium L. var. amara), grapefruit (Citrus paradisi), guarana (Paulinia cupanna), mate (Illex Paraguariensis), and cola nut (Cola Nitida), the method includes at least:
extracting (1a, 1b, 1c, 1d, 1e, 1f) polyphenols from blood orange (Citrus sinensis L. Osbeck) and sweet orange juice (Citrus aurantium L. var. sinensis L.), in order to obtain a first concentrate,
extracting (2a, 2b, 2c, 2d, 2e, 2f) polyphenols from by-products of blood orange (Citrus sinensis L. Osbeck), grapefruit (Citrus paradisi), sweet orange (Citrus aurantium L. var. sinensis L.) and bitter orange (Citrus aurantium L. var. amara), said extracting consisting of a separation of solid and liquid phases, said liquid phase being purified in order to obtain a second concentrate,
mixing (3a; 4a) the first and second concentrates obtained from the preceding steps of extraction to obtain a mixture,
after the step of mixing (3a; 4a), drying (3b; 4g) the mixture through atomizing on a maltodextrin carrier to obtain a product,
after the step of drying (3b; 4g), normalizing (3c; 4h) in caffeine, namely through addition of an extract of guarana (Paulinia cupanna), and/or mate (IIlex Paraguariensis), and/or cola nut (Cola Nitida) to the product resulting from the step of drying to obtain a resulting product,
then, finally, sieving (3d; 4i) the resulting product, in order to obtain a powder.

31. Method for obtaining a composition having lipolytic activity according to claim 30, wherein said mixture of plant species including at least 5 to 30 wt% blood oranges (Citrus sinensis L. Osbeck), 5 to 30 wt % grapefuits (Citrus paradisi), 5 to 25 wt % sweet oranges (Citrus aurantium L. var. sinensis L.), 1 to 10 wt % bitter oranges (Citrus aurantium L. var. amara), and 5 to 20 wt % guarana (Paulinia cupanna).

32. Method for obtaining a composition having lipolytic activity to claim 30, wherein the step of normalizing (3c; 4h) in caffeine is carried out through adding an extract of guarana (Paulinia cupanna) or of Mate (Illex Paraguariensis), or of Kola (Cola Nitida), to the product resulting from the step of drying (3b; 4g).

33. Method for obtaining a composition having lipolytic activity according to claim 30, wherein there is proceeded to the extraction (1a, 1b, 1e, 1d, 1e, 1f) of the anthocyanins and phenolic acids from juices of blood orange (Citrus sinensis L. Osbeck) and sweet orange (Citrus aurantium L. var. sinensis L.) and the extraction (2a, 2b, 2c, 2d, 2f) of flavanones from by-products of blood orange (Citrus sinensis L. Osbeck), grapefruit (Citrus paradisi), sweet orange (Citrus aurantium L. var. sinensis L.) and bitter orange (Citrus aurantium L. var. amara).

34. Method for obtaining a composition having lipolytic activity according to claim 30, wherein, previous to the step of drying (3b; 4g), a step of eliminating the water-insolubles particles from the mixture (4b, 4c, 4d, 4e, 4f) is conducted.

35. Use of the composition having lipolytic activity according to claim 23 as a food complement.

36. Use of the composition having lipolytic activity according to claim 23 to enrich food, namely beverages such as fruit and/or vegetable juices as well as milk products.

37. A method for enhancing breakdown of fat comprising administering the composition having lipolytic activity according to claim 23 to a patient.

38. A method for enhancing loss of body mass comprising administering the composition having lipolytic activity according to claim 23 to a patient.

39. A method for promoting lipolytic activity in a patient comprising administering the composition having lipolytic activity according to claim 23 in a posology between 1.0 and 2.0 grams, preferably 1.4 grams, of the composition per day to the patient.
